(19) United States
(12) Patent Application Publication
(10) Pub. No.: US 2012/0283509 A1
(43) Pub. Date: Nov. 8, 2012

(54) METHOD FOR INFLUENCING SEX SELECTION IN ARTIFICIAL INSEMINATION AND APPARATUS FOR SAME

(75) Inventor: Josef Pfistershammer, Claremont (AU)

(73) Assignee: BIASSEX Pty Ltd, West Perth, Western Austrailia (AU)

(21) Appl. No.: 13/511,924

(22) PCT Filed: Nov. 24, 2010
(86) PCT No.: PCT/AU2010/001576

§ 371 Date: Jul. 23, 2012

(30) Foreign Application Priority Data
Nov. 24, 2009 (AU) 2009905755
Jun. 25, 2010 (AU) 2010902816

(51) Int. Cl. A61B 17/43; A01N 1/02

(52) U.S. Cl. 600/35; 435/2

(57) ABSTRACT

A method for influencing sex selection in artificial insemination, the method characterised by the exposure of unsexed semen to antibodies to sex specific antigens present on spermatozoa carrying chromosomes of an undesired sex, whereby the spermatozoa to which the sex specific antigens are bound are generally unable to effect fertilisation of an egg, such that fertilisation is likely to be effected by spermatozoa carrying chromosomes of a desired sex.

### FIELD OF THE INVENTION

The present invention relates to a method for influencing sex selection in artificial insemination and apparatus for same. More particularly, the method of the present invention is intended to allow a user to skew the gender ratio, either simultaneously with or subsequent to, artificial insemination using unsexed semen.

### BACKGROUND ART

In livestock industries it is common to utilise artificial insemination for the genetic improvement, particularly in cattle. For example, it is possible to utilise the semen of a few highly selected males to inseminate thousands of females in any year.

For example, it may be desirable to bias the sex ratio in calf crops so as to produce more heifers in dairy herds. This enhances genetic selection pressure relating to production and confirmation of traits. It also allows for fewer calving difficulties, prevents the production of freemartins and bull calves, and enables longer lactation periods. Each of these results lead to more profits for the dairy herder.

A further basis on which to bias or skew the sex ratio within calf crops is to produce more male offspring in beef herds. This can enhance the quality and profitability of those herds or may be used to produce the next generation of herd bulls in intensively raised beef cattle.

In certain circumstances it is not possible to obtain sexed semen already packaged in straws. For example, sexed semen will not be available from quarantined, unproductive, or long dead sires. As such, it simply is presently not possible to obtain, in a convenient manner, sexed semen from such sires.

One object of the method and apparatus of the present invention is to overcome substantially the above mentioned problems of the prior art, or to at least provide a useful alternative thereto.

Each document, reference, patent application or patent cited in this text is expressly incorporated herein in their entirety by reference, which means that it should be read and considered by the reader as part of this text. That the document, reference, patent application, or patent cited in this text is not repeated in this text is merely for reasons of conciseness.

Reference to cited material or information contained in the text should not be understood as a concession that the material or information was part of the common general knowledge or was known in Australia or any other country.

Throughout this specification, unless the context requires otherwise, the word “comprise”, or variations such as “comprises” or “comprising”, will be understood to imply the inclusion of a stated integer or group of integers but not the exclusion of any other integer or group of integers.

### DISCLOSURE OF THE INVENTION

In accordance with the present invention there is provided a method for influencing sex selection in artificial insemination, the method characterised by the exposure of unsexed semen to antibodies to sex specific antigens present on spermatozoa carrying chromosomes of an undesired sex, whereby the spermatozoa to which the sex specific antigens are bound are generally unable to effect fertilisation of an egg, such that fertilisation is likely to be effected by spermatozoa carrying chromosomes of a desired sex.

Preferably, the spermatozoa carrying chromosomes of an undesired sex and to which the sex specific antigens are bound are unable to penetrate the Zona Pellucida surrounding the egg.

In one form of the present invention the sex specific antibodies are provided in the form of an adjunct, for example a powder.

Preferably, the exposure of the unsexed semen to the sex specific antibodies may occur during the physical act of artificial insemination.

Still preferably, this exposure occurs as the unsexed semen is injected into a uterus of a subject of artificial insemination.

In accordance with the present invention there is further provided a method for influencing sex selection in artificial insemination, the method characterised by the steps of:
(i) Positioning a biasing plug means in relation to a semen containing straw, such that as the unsexed semen contained within the straw is ejected it must pass through the biasing plug means; and
(ii) As the unsexed semen passes through the biasing plug means it is forced into intimate contact with sex specific antibodies provided in the biasing, plug means, those antibodies having been chosen to bind to sex specific antigens on spermatozoa carrying chromosomes of an undesired sex,
   whereby the intimate contact of the semen and the antibodies either in the biasing plug means or thereafter causes binding therebetween and by which the spermatozoa carrying chromosomes of the undesired sex and to which the sex specific antigens are bound are generally unable to effect fertilisation of an egg, thereby ensuring substantially that fertilisation is likely to be effected by spermatozoa carrying chromosomes of a desired sex.

In one form of the present invention the sex specific antibodies from within the biasing plug means are ejected therefrom into a uterus of an animal being inseminated along with the semen from the straw, the spermatozoa to which the sex specific antibodies are bound are then generally unable to effect fertilisation of an egg, thereby ensuring substantially that fertilisation may only be effected by spermatozoa carrying chromosomes of a desired sex.

Preferably, the biasing plug means comprises a casing defining therein a cavity, the cavity containing a volume of antibodies in the form of a powder.

Still preferably, the powder has provided therein a convoluted passage, the convoluted nature of which acts to ensure substantially that a significant portion of the antibody powder is ejected from the biasing plug means with the unsexed semen during the insemination process. The convoluted passage or canal may be provided in the form of a helix.

The biasing plug means is preferably positioned within an adaptor device. The adaptor device receives a cut end of the semen straw such that semen from the straw is directed through the biasing plug means.

In one form of the present invention for use in the artificial insemination of sows, the biasing plug means is provided within a joining device adapted to be positioned between a semen satchel and a catheter. The joining device is preferably generally cylindrical with open ends, over which removable seal means are provided. The seal means may each be provided in the form of heat-welded foil.

In accordance with the present invention there is further provided an apparatus for influencing sex selection in artificial insemination, the apparatus comprising a casing defining a cavity, the cavity having provided therein an amount of sex specific antibodies.

In one form of the present invention the sex specific antibodies are provided as a powder.

Preferably, the casing is formed of a mesh material. The pore size of the mesh material is preferably about 100 μm.

Preferably, the apparatus is configured such that it may be received on an artificial insemination straw in a manner that provides for ejected semen to pass through the apparatus.

Still preferably, the apparatus comprises a body defining a cavity in which the sex specific antibody powder is provided. The sex specific antibody powder may have a convoluted path defined therein. The convoluted path may be provided in the form of a helix.

In accordance with the present invention there is still further provided an artificial insemination sheath, the sheath characterised in that it comprises an adaptor device and biasing plug means as described hereinabove.

The sheath is preferably provided in a sealed package. The sealed packages may be provided in sheets wherein the packages are arranged side by side.

### BRIEF DESCRIPTION OF THE DRAWINGS

The method for influencing sex selection in artificial insemination and apparatus for same will now be described by way of example only, with reference to two embodiments thereof and the following drawings:

FIG. 1 is a cross-sectional side-elevational view of an apparatus for influencing sex selection in artificial insemination in accordance with the present invention; the apparatus shown in position within an adaptor by which the apparatus may be positioned within a sheath for bovine artificial insemination;

FIG. 2 is a cross-sectional side-elevational view of an apparatus in accordance with FIG. 1, the apparatus and adaptor shown in position on a semen straw and within a sheath of an artificial insemination gun;

FIG. 3 is a cross-sectional side-elevational view of an apparatus for influencing sex selection in artificial insemination in accordance with FIG. 1, the apparatus shown in position in a joining device for use in the artificial insemination of sows; and

FIG. 4 is a plan view of a package of multiple artificial insemination sheaths, each sheath being provided in an individual tear off compartment, and each sheath containing a single apparatus and adaptor in accordance with FIG. 1.

### BEST MODE(S) FOR CARRYING OUT THE INVENTION

The increase in population growth rate warrants the development of additional contraceptive methods that are widely acceptable, free from side effects and less expensive. Immuno-contraception, and in particular the targeting of antibodies to gamete-specific antigens implicated in sperm egg binding and fertilisation, offers an attractive approach to control fertility. The development of a contraceptive vaccine based on sperm antigen represents a promising approach to contraception. In mammals, fertilisation is completed by the direct interaction of sperm and egg, a process mediated primarily by sperm surface proteins. Sperm have sex specific proteins that are unique, cell specific, immunogenic and accessible to antibodies. Some sperm specific proteins have been isolated and characterized. The antibodies raised against the sperm specific antigens have proved to be extremely effective at reducing sperm-egg interaction in vitro. Fertility trials in sub-human primates would eventually prove the effectiveness of the sperm antigens in terms of contraceptive efficacy.

Currently, there are few inexpensive, reliable, effective methods for commercially separating X- and Y-chromosome bearing fresh and frozen bovine sperm. An experiment has been conducted by the Applicant to determine the efficacy of a commercially available post-thaw bovine semen sexing kit, HeiferPlus™ (HP) which claims to alter the sex ratio in favor of female calves following artificial insemination. Three trials were conducted, including the insemination of hyper-stimulated cows with Control or HP-treated semen, non-surgical embryo collection on Day 7, and a combined PCR/dot blot assay to determine embryo sex. Chi-square analysis showed that the Control group produced a greater proportion (p<0.0005) of female embryos than the HP group. There were no differences in the proportions of transferable, compared with degenerate, embryos or in number of ovulations, embryos, and unfertilized ova collected from Control compared with HP groups. When treatments were combined, one of the two bulls used in the hyperstimulation studies produced an overall greater proportion of females (p<0.05), suggesting a bull effect.

The only marginally successful semen sexing of the prior art is based on Flow Cytometry. Flow Cytometry is a technique used to sort and classify cells using fluorescent markers on their surface and passing them by an electronic detection apparatus, such as a Laser beam. Not only is the equipment used extremely expensive, but it is also very slow and the yield is only 25% male, 25% female, the remainder (i.e. 50%) is unsorted, sub-fertile semen.

The evolutionary development of the Y chromosome in mammals has resulted in it carrying large amounts of genetic information. Of this large amount of genetic information some is unique to the male chromosome. Nearly all genetic material on the X chromosome is a copy of itself many times over. Further, almost all of the functional genomic material on the X chromosome has its homologues on the Y chromosome. Thus, it is highly improbable that sex specific antigens will be located on X carrying sperm surfaces. This then means, immunologically, that it is most likely that only male sperm can be incapacitated, negating the prior art attempts to select against female sperm, or to sorting an ejaculate into both sexes through the use of immunological principles.

As may be noted by the foregoing, the mechanics and biology of artificial insemination in livestock industries is well established and understood, including the use of semen contained in straws. For example, commercially available semen in Australia is packed in coloured straws, coded according to individual breeds. Each straw has printed thereon a range of information needed to identify an individual bull.

Despite the generally advanced state of understanding regarding the mechanics and biology of artificial insemination it is not presently possible to, with any degree of accuracy, utilise unsexed semen already contained in straws and to skew or bias the sex of semen from those straws during the artificial insemination process. The consequence being that the unsexed semen of quarantined, unproductive, or long dead sires cannot be utilised in circumstances requiring substantially sexed semen.

U.S. Pat. No. 4,999,283 discloses “a method for separating male and female determining spermatozoa” in which semen is exposed to an “excess concentration of monoclonal antibody directed against” antigens that are specific to spermatozoa that determine one sex or the other, or sex specific membrane proteins. The entire content of U.S. Pat. No. 4,999,283 is incorporated herein by reference.

Using the method disclosed in U.S. Pat. No. 4,999,283 as a starting point, the present Applicant provides a method that facilitates biasing or skewing of unsexed semen from straws during the artificial insemination process, without the need for substantial modification of the techniques for such presently utilised in those industries employing artificial insemination.

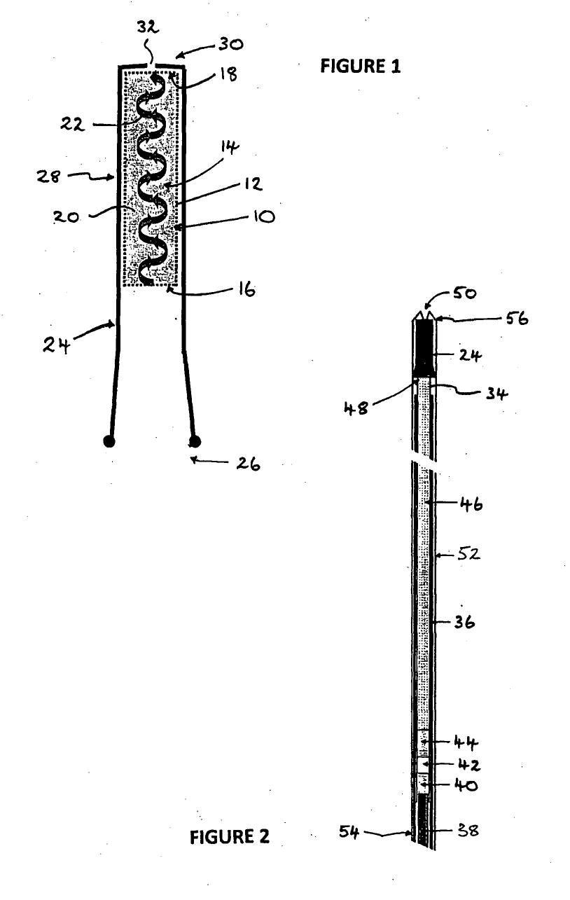

In FIG. 1 there is shown an apparatus for influencing sex selection in artificial insemination, or a biasing plug means 10, in accordance with a first embodiment of the present invention. The biasing plug means 10 comprises a casing 12 defining therein a cavity 14 extending through the body 12 from a first end 16 thereof, to a second end 18 thereof.

The cavity 14 has provided therein an adjunct, for example an amount of powdered sex specific antibody 20, the amount thereof being sufficient so as to allow binding with, or agglutination of, substantially all of the male spermatozoa during the artificial insemination process.

A convoluted passage 22 is provided extending through the powdered antibody 20. The passage 22 extends from the first end 16 to the second end 18. The provision of this convoluted passage 22, for example in the helical form depicted, ensures substantially that as the unsexed semen is forced through the biasing plug means 10 that pressure is exerted on the powdered antibodies 20 therein which acts to force the powdered antibodies 20 into a uterus (not shown) of an animal being artificially inseminated. The passage 22 is relatively narrow at the first end 16 of powdered antibody 20 but widens somewhat as it progresses towards the second end 18. This widening of the passage 22 is intended to ensure that substantially all the powdered antibody 20 is expelled and that agglutination of sperm and antibodies within the biasing plug means 10 doesn't impede the process.

The casing 12 is formed of a mesh, for example a nylon mesh having a pore size of about 100 μm. The casing is provided in generally cylindrical form which appears rectangular in section. In one form of the present invention the biasing plug means 10 is about 2 mm in width/diameter and about 8 mm in length, this being the form most commonly used in bovine applications.

The biasing plug means 10 of FIG. 1 is shown located within an adaptor device 24. The adaptor device 24 has a generally cylindrical form having a first end 26, an intermediate portion 28 and a second end 30. The first end 26 is open and of slightly broader circumference than the remainder of the adaptor device 24. The intermediate portion 28 is arranged to accommodate the biasing plug means 10 such that the biasing plug means fits snugly therein, as shown in FIG. 1. The second end 30 is closed apart from a single central aperture 32.

In FIG. 2 there is shown the biasing plug means 10 and its adaptor device 24 in position on a semen straw 34 of generally known type. The straw 34 is in turn received within a nozzle 36 of an artificial insemination gun (shown only in part). A steel plunger 38 of the gun acts on a cotton wad 40 provided at the base of the straw 34. A powder plug 42 is in turn provided above the cotton wad 40. A further cotton wad 44 is provided above the powder plug 34. Above the cotton wad 44 there is provided a volume of unsexed semen 46. The biasing plug means 10 and adaptor device 24 are positioned at a cut end 48 of the semen straw 34, whereby the cut end 48 of the semen straw 34, on which the biasing plug means 10 is positioned, is able to interact in a functional manner with a mouth 50 of an artificial insemination sheath 52.

The artificial insemination sheath 52 is elongate and cylindrical in form and is of a diameter such that the adaptor device 24 fits snugly within it. The artificial insemination sheath 52 has a first end 54 and a second end 56. The first end 54 is provided in an open configuration whereas the second end 56 is generally closed, apart from the centrally located mouth 50 described above.

The volume of semen 46 is able to pass from the cut end 48 of the semen straw 34 into and through the biasing plug means 10, in turn through the aperture 32 of the adapter device 24, from which the semen 46 passes to the mouth 50 of the artificial insemination sheath 52.

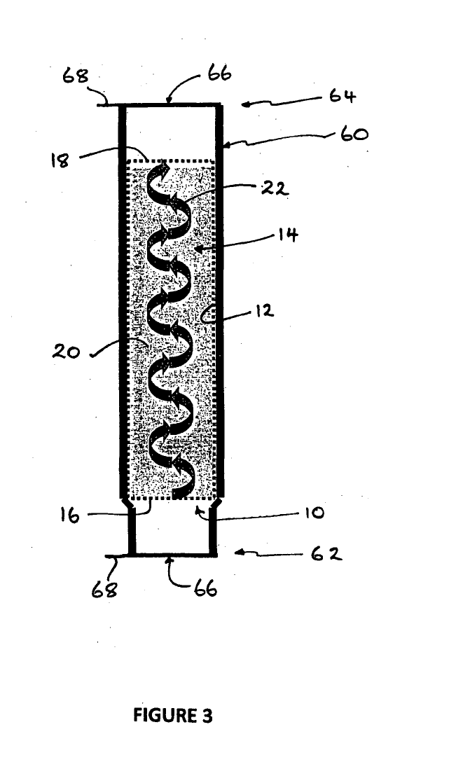

In FIG. 3 there is shown the biasing plug means 10 in position within a joining device 60 for use when artificially inseminating sows (not shown). The joining device 60 is proportioned to be inserted between a semen satchel and a catheter (neither shown) such as are used when artificially inseminating sows.

The joining device 60 is of a generally cylindrical configuration, having a first open end 62 and a second open end 64. The biasing plug means 10 is positioned within the joining device, intermediate the ends 62 and 64, as shown in FIG. 3. Each end 62 and 64 has provided thereover a removable seal means, for example heat welded foil seals 66 having tabs 68 provided thereon and whereby a user (not shown) may grip the tab and remove that seal 66. The seals 66 act to seal against moisture intrusion and contamination.

The biasing plug means 10 when used for inseminating sows is much larger than its equivalent used in bovine insemination. For example, a width/diameter of about 3.9 mm and a length of 12 mm is preferred, due to the high volume of semen used in the insemination of sows.

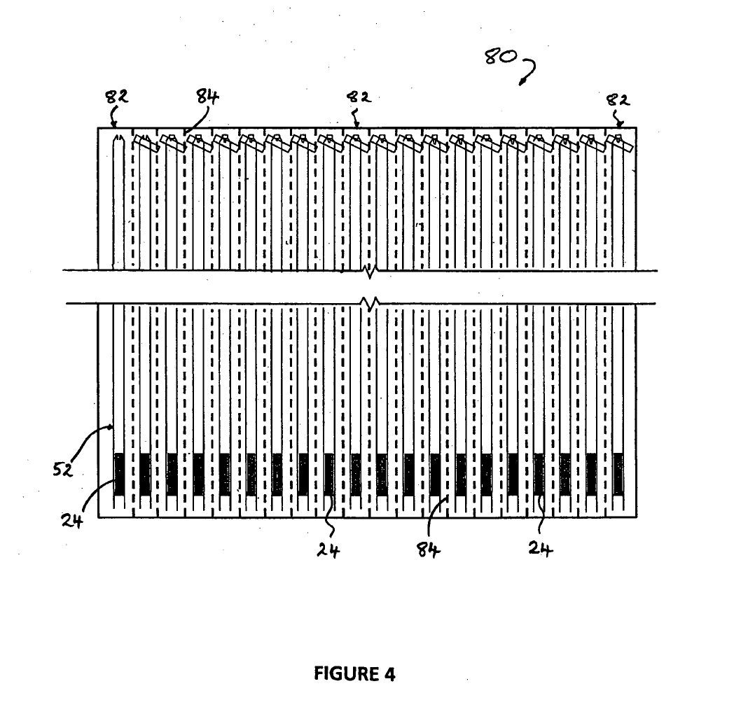

In FIG. 4 there is shown a sheet 80 of twenty (20) artificial insemination sheaths 52. Each sheath 52 has provided therein a single adaptor device 24, in which is housed a biasing plug means 10 (not visible in FIG. 4). The adaptor device 24 is positioned adjacent the first end 54 of the sheath 52 at first instance, it being the interaction of the sheath 52 and biasing plug means 10 with the straw 34 that pushes the adaptor device 24 to the second end 56 of the sheath 52 (the position shown in FIG. 2).

The sheet 80 comprises a plurality of sealed packages 82, each package 82 containing a single sheath 52. The packages 82 are joined to that package adjacent to it by a tear-off or die-cut join 84, whereby individual packages 82 may be removed by a user (not shown) as required.

In use, a user (not shown) wishing to artificially inseminate an animal using unsexed semen from a semen straw 34 will follow the typical protocols when undertaking typical artificial insemination procedures. However, if, for example, the user wishes to skew the sex of any progeny towards females, the user will select a biasing plug means 10 in which the antibody powder is specific polyclonal antibody that will agglutinate semen containing the Y chromosome. That is, if a female progeny is desired then polyclonal antibodies specific for “male sperm” are required. The following discussion will be conducted on the basis that the user desires female progeny.

Accordingly, the user tears off a package 82 from the sheet 80, the package containing a sheath 52 and its contained adaptor device 24 and the biasing plug means 10 therein. The package 82 is opened and the sheath removed.

The semen straw 34 and nozzle 36 of the insemination gun to which it has been fitted in know manner is then inserted into the sheath 52 such that the cut end 48 of the straw 34 pushes into the adaptor device. The gun and straw subsequently function as they would normally during operation. However, the unsexed semen 46 from the straw 34 are pushed against the first end 16 of the casing 12, from which it enters the passage 22. The convoluted nature of the passage 22 ensures that the force that is being exerted by the semen 46 acts against the powdered antibody 20 causing same to be pushed through the mesh of the casing 12, through the aperture 32 of the adaptor device 24, through the mouth 50 of the sheath 52 and into a uterus of the animal being inseminated. In this manner a combination of the powdered antibody 20 and the unsexed semen is injected into the animal in intimate contact, at which time the antibodies and semen may interact in the manner described hereinafter.

This allows time for the antibodies to bind to the antigens present on the male spermatozoa which in turn then incapacitates the male spermatozoa such that they are incapable of penetrating the Zona Pellucida of the egg. Such male spermatozoa are similarly not able to attach or bind to the Zona Pellucida, nor can they shed their acrosome. Accordingly, they cannot squeeze through the tight layer of cells surrounding the egg.

If during the fertilization process only sperm carrying the desired sex chromosome can interact with the egg then only progeny of the wanted gender will be generated. For fertilisation to occur, a sperm must first make its way through two outer layers of the egg; the cumulus and the Zona Pellucida.

To do this, the sperm head undergoes an acrosomal reaction in which enzymes located in the sperm's acrosome digest the cumulus and the Zona Pellucida layers. The sperm then reaches the egg's vitelline envelope where bindin protein on the sperm reacts with bindin receptors on the envelope. When these proteins recognise each other, the egg membrane swallows the sperm head. This allows the sperm nucleus to enter the egg's cytoplasm and fuse with the egg nucleus The neck and tail are excluded.

One underlying principle of the present invention is to incapacitate the undesired sperm with antibodies so they are incapable of progressing through the Zona Pellucida of the egg. The incapacitated sperm are not able to attach to the Zona Pellucida, nor can they bind to it or shed their acrosome (the “acrosome reaction”). Accordingly, the incapacitated sperm cannot squeeze through the tight layer of cells surrounding the ovum to fuse with it.

The acrosome reaction is the process of activation for fertilisation, just prior to penetration of the sperm's DNA into the egg. In this reaction, enzymes are released and surface antigens are exposed as the anterior part of the sperm ruptures, allowing fertilisation to proceed. This essential process cannot occur if sex specific antibodies (ASAs) are attached to surface antigens.

The perivitelline space is the fluid-filled area between the egg and yolk membranes. A sperm must undergo the acrosome reaction in order to enter this section of the egg to eventually fertilise it. A sperm encumbered as described above cannot achieve this essential penetration feat.

As can be seen, the incapacitation of the undesired sperm stops such sperm from penetrating through to the egg due to their inability to shed the acrosome and release a vital enzyme. Accordingly, the DNA of the undesired sperm cannot combine with that of the ovum to create an embryo. This is an immunological protection of the zygote from undesirable, or potentially harmful proteins.

As further principle of the present invention, or as additional “insurance”, the present invention makes use of the fact that antibodies provide the linkage between the dozens of sex specific peptides (i.e. antigens) on the sperm surface and the “encumbering” extra large (i.e. heavy) baggage proteins (i.e. antibody+protein). This extra weight is solidly attached to the antigen and the latter cannot rid itself of this encumbrance, preventing the sperm from successfully competing for the fusion with the egg.

The Applicants have found that through use of the method and apparatus of the present invention the sex of the offspring may be skewed with 85 to 90% accuracy. Accordingly, it is now possible to, with a degree of accuracy, utilise unsexed semen already contained in straws and to skew or bias the sex of semen from those straws during the artificial insemination process. The consequence being that the unsexed semen of quarantined, unproductive, or long dead sires can, using the method and apparatus of the present invention, be utilised in circumstances requiring substantially sexed semen.

Modifications and variations such as would be apparent to the skilled addressee are considered to fall within the scope of the present invention. For example, it is envisaged that the powdered antibody referred to hereinabove and which is used to bind with antigens present on the surface of male spermatozoa may be exposed to unsexed semen in a range of manners not specifically described hereinabove. For example, the antibody and unsexed spermatozoa may be combined in the semen straw, or in a process prior to loading of the semen into the artificial insemination straw.

1. A method for influencing sex selection in artificial insemination, the method characterised by the exposure of unsexed semen to antibodies to sex specific antigens present on spermatozoa carrying chromosomes of an undesired sex, whereby the spermatozoa to which the sex specific antigens are bound are generally unable to effect fertilisation of an egg, such that fertilisation is likely to be effected by spermatozoa carrying chromosomes of a desired sex.

2. The method according to claim 1, wherein the spermatozoa carrying chromosomes of the undesired sex and to which the sex specific antigens are bound are unable to penetrate the Zona Pellucida surrounding the egg.

3. The method according to claim 1, wherein the sex specific antibodies are provided in the form of an adjunct, for example a powder.

4. The method according to claim 1, wherein the exposure of the unsexed semen to the sex specific antibodies occurs during the physical act of artificial insemination.

5. The method according to claim 4, wherein the exposure occurs as the unsexed semen is injected into a uterus of a subject of artificial insemination.

6. A method for influencing sex selection in artificial insemination, the method characterised by the steps of:
(i) Positioning a biasing plug means in relation to a semen containing straw such that as the unsexed semen contained within the straw is ejected it must pass through the biasing plug means; and
(ii) As the unsexed semen passes through the biasing plug means it is forced into intimate contact with sex specific antibodies provided in the biasing plug means, those antibodies having been chosen to bind to sex specific antigens on spermatozoa carrying chromosomes of an undesired sex male spermatozoa,
whereby the intimate contact of the semen and the antibodies either in the biasing plug means or thereafter causes binding therebetween and by which the spermatozoa to which the sex specific antigens are bound are generally unable to effect fertilisation of an egg, thereby ensuring substantially that fertilisation is likely to be effected by spermatozoa carrying chromosomes of a desired sex.

7. The method according to claim 6, wherein the sex specific antibodies from within the biasing plug means are ejected therefrom into a uterus of an animal being inseminated along with the semen from the straw, the spermatozoa to which the sex specific antibodies are bound are then generally unable to effect fertilisation of an egg, thereby ensuring substantially that fertilisation may only be effected by spermatozoa carrying chromosomes of the desired sex.

8. The method according to claim 6, wherein the biasing plug means comprises a casing defining therein a cavity, the cavity containing a volume of antibodies in the form of a powder.

9. The method according to claim 8, wherein the powder has provided therein a convoluted passage, the convoluted nature of which acts to ensure substantially that a significant portion of the antibody powder is ejected from the biasing plug means with the unsexed semen during the insemination process.

10. The method according to claim 9, wherein the convoluted passage is provided in the form of a helix.

11. The method according to claim 6, wherein the biasing plug means is positioned within an adaptor device.

12. The method according to claim 11, wherein the adaptor device receives a cut end of the semen straw such that semen from the straw is directed through the biasing plug means.

13. The method according to claim 6, wherein the biasing plug means is provided within a joining device adapted to be positioned between a semen satchel and a catheter in the artificial insemination of sows.

14. The method according to claim 13, wherein the joining device is generally cylindrical with open ends, over which removable seal means are provided.

15. The method according to claim 14, wherein the seal means are each provided in the form of heat-welded foil.

16. An apparatus for influencing sex selection in artificial insemination, the apparatus comprising a casing defining a cavity, the cavity having provided therein an amount of sex specific antibodies.

17. The apparatus according to claim 16, wherein the sex specific antibodies are provided as a powder.

18. The apparatus according to claim 16, wherein the casing is formed of a mesh material.

19. The apparatus according to claim 18, wherein the pore size of the mesh material is about 100 μm.

20. The apparatus according to claim 16, wherein the apparatus is configured such that it may be received on an artificial insemination straw in a manner that provides for ejected semen to pass through the apparatus.

21. The apparatus according to claim 16, wherein the sex specific antibody powder may have a convoluted path defined therein.

22. The apparatus according to claim 21, wherein the convoluted path is provided in the form of a helix.

23. The apparatus according to claim 16, wherein the apparatus is received with an adaptor device, the adaptor device being proportioned to receive a cut end of a semen straw such that semen from the straw is directed through the biasing plug means.

24. An artificial insemination sheath, the sheath characterised in that it is cylindrical in form and has an adaptor device and apparatus for influencing sex selection in artificial insemination provided therein, the adaptor device is proportioned to receive a cut end of a semen straw such that semen from the straw is directed through the apparatus.

25. The artificial insemination sheath according to claim 24, wherein the apparatus is as defined in any one of claims 16 to 22.

26. The artificial insemination sheath according to claim 24, wherein the sheath is provided in a sealed package.

27. The artificial insemination sheath according to claim 26, wherein the sealed packages may be provided in sheets wherein the packages are arranged side by side.

28. (canceled)

29. (canceled)
